(19) United States
(12) Patent Application Publication
(10) Pub. No.: US 2026/0160762 A1
(43) Pub. Date: Jun. 11, 2026

(54) BIODEGRADABLE ANALYTE DETECTION DEVICE

(71) Applicant: HOOPSY LTD, London (GB)

(72) Inventors: Lara Solomon, London (GB); Cortina McCurry, Melbourne (AU)

(21) Appl. No.: 19/305,992

(22) Filed: Aug. 21, 2025

Related U.S. Application Data
(63) Continuation of application No. PCT/AU2024/050156, filed on Feb. 27, 2024.
(60) Provisional application No. 63/449,041, filed on Feb. 28, 2023.

(51) Int. Cl. G01N 33/543

(52) U.S. Cl. CPC G01N 33/54391

(57) ABSTRACT

A device for detecting an analyte in a sample is provided. The device comprises a test strip fluid including a sample collection pad for contacting the sample. A conjugate release pad is in fluid communication with the sample collection pad. The conjugate release pad comprises at least one labelling reagent, wherein the at least one labelling reagent comprises a component capable of binding to the analyte at a first location. The test strip comprises a test line such that the analyte binds at a second location to a test antibody contained within the test line.

### TECHNICAL FIELD

The present invention relates to a device for detecting an analyte in a sample; and, more particularly, to a device for detecting an analyte in a sample that does not require medical or technical training to operate, and is made from biodegradable material.

### BACKGROUND

Simple lateral flow immunoassay devices are known and commonly available for detection of analytes in fluid samples. Self-administered home pregnancy tests are an example of such a device and are sold globally at an estimated 20 million pregnancy tests per year¹. Expanding at a compound annual growth rate of 5.2%, the global Fertility and Pregnancy Rapid Test Kits Market is projected to increase from an estimated valuation of US$1.4 billion in 2022 to US$2.1 billion by 2032². In terms of environmental impact, globally 1.6 billion lateral flow test kits are sold per year, weighing on average 10-15 grams, this results in 25 million kilograms of plastic pollution for that year alone³. Once in landfill, the polypropylene in a typical rapid test kit will slowly degrade and can take anywhere from 20-30 years to completely breakdown. This poses significant environmental concern because the additives used in polypropylene manufacture may include toxins like cadmium and lead³. ¹ www.bioamd.com/investors#:˜:text=There is a growing migration, are sold globally each year.² https://www.marketresearchfuture.com/reports/pregnancy-test-kits-market-2982³ https://www.aaapolymer.com/a-simple-guide-to-polypropylene-recycling-for-businesses/#:˜:text=Once%20in%20landfills%2C%20polypropylene%20slowly,toxins%20like%20cadmium%20and%20lead

As illustrated by their popularity, lateral flow assay devices such as rapid pregnancy test kits are easy to use, low cost, and allow for rapid diagnostics to be performed. These devices are easy to manufacture and there is a growing interest in diagnostic testing from governments and healthcare systems suggesting that these numbers will increase over the coming years. Existing lateral flow assay devices such as pregnancy test kits have changed minimally in design from the introduction of a dipstick lateral flow assay in the 1950s. Lateral flow assay devices tend to be rigid in structure and made from non-biodegradable materials. Additionally, they have a flat, rectangular and linear design which is reliant on capillary action to move samples from the sample collection area through the device to the testing area. While a flat and rectangular shape contributes to capillary action and the flow of fluid from the sample collection area to the testing area, users find the current designs to be awkward and unhygienic to use.

Urinating into a cup and subsequently inserting a test into the fluid is seen as cumbersome and unhygienic and 95% of women prefer to use a midstream test⁴. In the case of midstream tests, it is difficult to control the flow of fluid being collected, as a user is required to urinate over the top of a rectangular wick. There is no mechanism for fluid retention beyond what is absorbed through wick. The existing designs can be very difficult to use from the perspective of optimal sample collection and volume control. ⁴https://www.ncbi.nlm.nih.gov/pmc/articles/PMC4119102

There is very little control over the timing of fluid flow, or reactions occurring at various stages of the device.

Simple lateral flow immunoassay devices have been developed and commercialised for detection of analytes in fluid samples. An example of such a device is disclosed in EP291194. Such devices typically comprise a porous carrier comprising a dried, mobilisable labelled binding reagent capable of binding to the analyte in question, and an immobilised binding reagent also capable of binding to the analyte provided at a detection zone downstream from the labelled binding reagent. Detection of the immobilised labelled binding reagent at the detection zone provides an indication of the presence of analyte in the sample.

Existing prior art devices are illustrated in FIG. 1. As shown in FIG. 1a, existing devices have a sample pad A is to accept the fluid sample for testing. The conjugate release pad B hosts one or more mobile label reagents such as gold nanoparticles or latex beads, etc, that recognize, or are capable of binding the analytes of interest. A membrane C hosts the test line D and control line E, which is usually made of non-biodegradable nitrocellulose. An adsorbent pad F captures excess fluid. A backing card G, also non-biodegradable, provides physical support for the test.

Determination of the result of the assay has been traditionally carried out by eye. However, the user is required to interpret the results from these devices and this can introduce an undesirable degree of subjectivity, particularly at low analyte levels when the intensity of the analyte detection is weak. Digital devices have therefore been developed to determine the result of the assay using an optical detection means as well as a display means to display the result of the assay. Digital assay readers are known to be used in combination with assay test-strips for determining the concentration and/or amount of analyte in a fluid sample. Assay devices comprising an integral digital assay reader are also known. An example of such a device is disclosed in EP 1484601. Light from a light source, such as a light emitting diode (LED), is shone onto a portion of the porous carrier and either reflected or transmitted light is detected by a photodetector. Typically, the reader will have more than one LED to illuminate various zones of the carrier, and a corresponding photodetector is provided for each of 20 the plurality of LEDs. EP1484601 discloses an optical arrangement for a lateral flow test strip digital reading device comprising a baffle arrangement allowing for the possibility of reducing the number of photodetectors in the device.

European Patent 3332255B1 discloses a diagnostic device comprising a test strip encased within a support, wherein the test strip and the support are each comprised of a water-dispersible matrix material, characterized in that the water-dispersible matrix-material is a nonwoven web material; and the support is treated with a hydrophobic coating. The test strip comprises a test zone and is in fluid communication with a sample zone and an absorbent zone, wherein the sample zone and the absorbent zone are comprised of a water-dispersible material. The support comprises one or more slits or one or more cut outs through the matrix material to enhance wettability, wherein the one or more slits or the one more cut-outs provide greater access to an internal matrix structure by a liquid, and reduces liquid surface tensions when the device is contacted with a body of liquid. The device comprises an antibody reagent comprising a sugar comprising trehalose and sucrose wherein the antibody is releasably deposited on the test strip wherein the antibody is specific for an analyte. However, the invention of EP3332255B1 does not address the issues of ease of use and fluid volume control during sample capture and embodiments of the device rely primarily on capillary action for transfer of the sample to the test zone.

U.S. Pat. No. 5,611,995 discloses an apparatus that detects a specifically reacting substance in a test liquid. The apparatus has a housing and a holding device for holding a test strip. The test strip has a material that transports a test liquid essentially by capillary forces and has an analytical system which indicates the presence or absence of the substance to be detected. The holding device can be attached to the housing with an opening for allowing evaporation of test liquid. The housing can be elongated for accepting a sample collector therein. A contact mechanism can also be disposed for promoting contact of liquid sample from the sample collector when inserted in the housing, to the test strip held by the holding device. However, the invention of U.S. Pat. No. 5,611,995 does not address the issues of ease of use and fluid volume control during sample capture and embodiments of the device rely primarily on capillary action for transfer of the sample to the test strip. Further, no mention is made regarding the biodegradability or flushability of the apparatus.

U.S. Pat. No. 8,268,636 discloses a device for detecting an analyte in a liquid sample deposited on a first portion of the device for transport to a second portion of the device that is in fluid contact with the first portion. In specific embodiments, the device comprises a labelled conjugate comprising a binding member reactive with a first epitope of the analyte and a label comprising a gold colloid, preferably having a mean particle size of 50 nm to 100 nm. In further embodiments, the device comprises a capture component comprising polymerized streptavidin. However, the invention of U.S. Pat. No. 8,268,636 does not address the issues of ease of use and fluid volume control during sample capture. Embodiments of the device rely primarily on allowing the liquid sample to “flow across the first portion and the second portion of the substrate” for transfer of the sample to the test zone. Further, no mention is made regarding the biodegradability or flushability of the apparatus.

Any discussion of the prior art throughout the specification should in no way be considered as an admission that such prior art is widely known or forms part of common general knowledge in the field.

### SUMMARY

### Problems to be Solved

Challenges experienced by end users when using a lateral flow test, such as a “self-test” pregnancy test include insufficient sample fluid capture wherein too much or too little sample leads to erroneous results. Insufficient migration of the sample fluid on the collection pad to test area leads to poor quality results with a high number of false positives, and false negatives. There can also be user confusion in determining where to place the sample, or how to read the results obtained. In the case of a pregnancy test, there can be user confusion in determining whether it is a positive or negative result.

Existing plastic test devices contribute to landfill when disposed of as lateral flow immunoassay tests are typically made of hard and rigid materials that are required to be stable and non-biodegradable due to the functionality required by the device. Many tests on the market use glass fibres for the conjugate pad which can present a health hazard in automated manufacturing.

Key aspects of existing technology are illustrated in FIG. 1, showing drawings of the prior art existing technologies. In FIG. 1a of the prior art drawings, the existing pregnancy test device is a flat and linear design, lacking dimensionality and relying primarily on capillary action. The sample pad and conjugate release pad are made of non-biodegradable materials.

Further challenges with existing urine-based lateral flow tests include small and flat absorbent wicks which frequently lead to null/ambiguous results due to insufficient sample collected. The user may not keep the test within the flow of urine for long enough and a small, flat absorbent wick means that urine passes over but is not retained, preventing control over liquid volume. The user often does not hold the test at the appropriate angle to capture the sample flow.

Referring to Prior Art Drawings FIG. 1b, existing strip test designs requires two steps, first collecting a urine sample and then placing the test strip into the fluid. This can be an unhygienic and messy process and many users do not want to handle urine. Some users prefer a midstream test

Referring to Prior Art Drawings FIG. 1c, existing cartridge test designs are labour intensive and required the inconvenience of using a dropper to drip the urine into the test well. Users are often confused about where to place the urine sample and frequently make mistakes and drip the urine into the test window, where the results show up. Similar to strip tests, existing cartridge test are perceived as unhygienic by users.

Referring to Prior Art Drawings FIG. 1d, existing midstream urine tests with a cap have been developed in an effort to retain sample liquid and prevent dripping: However, users are often nervous and uncomfortable and neglect to remove the cap before urinating on the test stick.

It is an aim and objective of the present invention to provide a device for detecting an analyte in a sample that is comfortable for a user to operate as well as sensitive, specific, and readable.

It is an aim and objective of the present invention to provide a device for detecting an analyte in a sample which provides effective sample collection and labelling, along with rapid result availability, analyte sensitivity, and accuracy.

It is an aim and objective of the present invention to provide a device for detecting an analyte in a sample which provides user control over fluid volume and the timing of fluid flow.

It is an aim and objective of the present invention to provide a device for detecting an analyte in a sample which can be discrete for a user to operate, and to dispose of.

It is an aim and objective of the present invention to provide a device for detecting an analyte in a sample wherein the device is made from material that is biodegradable, compostable, and in some variations, flushable.

It is an object of the present invention to overcome or ameliorate at least one of the disadvantages of the prior art, or to provide a useful alternative.

### Means for Solving the Problem

According to a first aspect, the invention provides a device for detecting an analyte in a sample; the device comprising a test strip fluid including a sample collection pad for contacting said sample; a conjugate release pad in fluid communication with the sample collection pad, wherein the conjugate release pad comprises at least one labelling reagent, wherein the at least one labelling reagent comprises a component capable of binding to the analyte at a first location; wherein the test strip comprises a test line such that the analyte binds at a second location to a test antibody contained within the test line.

Preferably, the device further includes a handle, the test strip extending through the handle; and a hemispherical sample collection reservoir connected to the handle; wherein the sample collection reservoir is designed to retain fluid for contact with the sample collection pad located within the sample collection reservoir.

Preferably, the hemispherical sample collection reservoir is in the shape of a spoon or ladle.

Preferably, the test strip comprises a cellulose membrane and the sample collection pad and the conjugate release pad comprise a biodegradable blended cellulose membrane. The test antibody preferably comprises a custom antibody, such as a monoclonal, single-chain variable fragment or camelid nanobody, fused to a molecule

with an affinity to cellulose. Preferably, the custom antibody is produced by fusing cellulose binding molecules such as carbohydrate-binding molecules (CBMs) to antibody fragments to increase protein binding capacity. The protein bonding capacity of CBM-fused antibody fragments compared to sole antibody fragments is preferably increased by a factor of between 2 and 5.

Preferably, the sample collected in the sample collection reservoir is directed through at least one vertical fluid flow path and at least one horizontal fluid flow path in the sample collection pad and/or the conjugate release pad prior to passing through to the test strip within the handle of the device. The at least one vertical fluid flow path and the at least one horizontal fluid flow path may be hardened or embossed to direct flow of fluid through the device. A speed and a direction of fluid flowing through the at least one vertical fluid flow path and the at least one horizontal fluid flow path can be controlled by integration of fabricated channels with borders composed of a non-toxic material such as a wax or plant-based glue to guide the direction of fluid.

The device can be made can be made from material comprising unbleached wood pulp and viscose blend to maintain dispersibility, and reducing sample flow by interaction of the sample with hydroxyl groups on unbleached cellulose fibres.

Preferably, polyvinyl chloride (PVC) or polydimethylsiloxane can be used to plug cellulose membrane pores in the test strip to reduce a lateral flow of the analyte thereby increasing interaction time between the analyte and the test antibody.

The conjugate release pad is contained within the hemispherical sample collection reservoir in a layer beneath the sample collection pad, such that the conjugate release pad is rehydrated before binding to the analyte in the sample and flowing with the sample into the test strip in the handle. Alternatively, the conjugate release pad is contained within the handle of the device to interact with the sample before flowing into the test strip.

Preferably, the test strip further comprises a control line wherein the control line comprises the test antibody that is capable of binding to an unbound component of the labelling reagent. The labelling reagent preferably comprise colloidal gold, latex beads or cellulose nanobeads.

Preferably, the handle and the sample collection reservoir are made from materials that are biodegradable, compostable and/or flushable such as unbleached wood pulp, viscose, potato, sugar cane, wheat straw fibre, banana fibres, corn-starch or other biodegradable materials. Sugar, gelatine or wax may be used to provide rigid support to the handle and waterproof backing of the device or to coat the sample collection reservoir to control the direction of fluid flow.

One or more holes may be located around the perimeter of the device to serve as wells that are fillable with a dissolvable substance to facilitate dispersion of the device when flushed. The handle of the device may be detachable from the sample collection reservoir such that the sample collection reservoir can be detached, dissolved and disposed of, or wherein where a modular test strip can be included into the handle of the device.

The test strip may be configured to detect an analyte comprising human chorionic gonadotropin (hCG). Preferably, the test antibody is designed as a custom antibody that is sensitive to the beta subunit of hCG, or hyperglycosylated hCG, or pregnanediol glucuronide (PdG), or other such hormones capability of detection characteristics specific to fertility. Some embodiments may comprise multiple test antibodies to hCG such that pregnancy can be monitored by week, or to enable verification of the number of weeks of pregnancy at the time of the test.

Analytes which may be tested by the device include, but are not limited to, glucose, ketones, pH levels, bilirubin, nitrites/leukocyte esterase, alcohol, amphetamines, benzodiazepines, cannabis, cocaine, opioids, vitamin D, pathogens, soil analysis, pesticide detection, lead, plant health tests, Legionella, menopause, thyroid, and enzyme indicators.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1a to FIG. 1d are perspective, front and top views of examples of prior art of the invention;

FIG. 2 shows top views of a preferred embodiment of the invention;

FIG. 3 is a top view of a preferred embodiment of the invention showing the test line and a control line on the test strip of the device.

FIG. 4A and FIG. 4B are alternative side views of a preferred embodiment of the invention depicting different locations of the conjugate release pad.

FIG. 5 is a top view of a preferred embodiment of the invention depicting an example of the use of wax or embossing in the channels to support fluid flow.

FIG. 6 shows top views of a preferred embodiment of the invention depicting a polyvinyl chloride (or similar) solution dotted or coated on the test strip to support plugging of the pores thereon.

FIG. 7 is a top view of a preferred embodiment of the invention depicting an example of the use of wells around the perimeter of the device that can be filled with a dissolvable substance to facilitate dispersion/flushability of the device.

FIG. 8 is a side view of a preferred embodiment of the invention depicting a device that is foldable and may be detached from the handle, dissolved and disposed of.

FIG. 9a, 9b and 9c are perspective, top and side views of a preferred embodiment of the invention showing vertical and horizontal flow paths.

FIG. 10 shows another preferred embodiment of the invention.

FIGS. 11a and 11b shows another preferred embodiment of the invention in a flat configuration (FIG. 11a) and a folded in-use configuration (FIG. 11b).

FIG. 12 shows another preferred embodiment of the invention having a dissolvable collection reservoir.

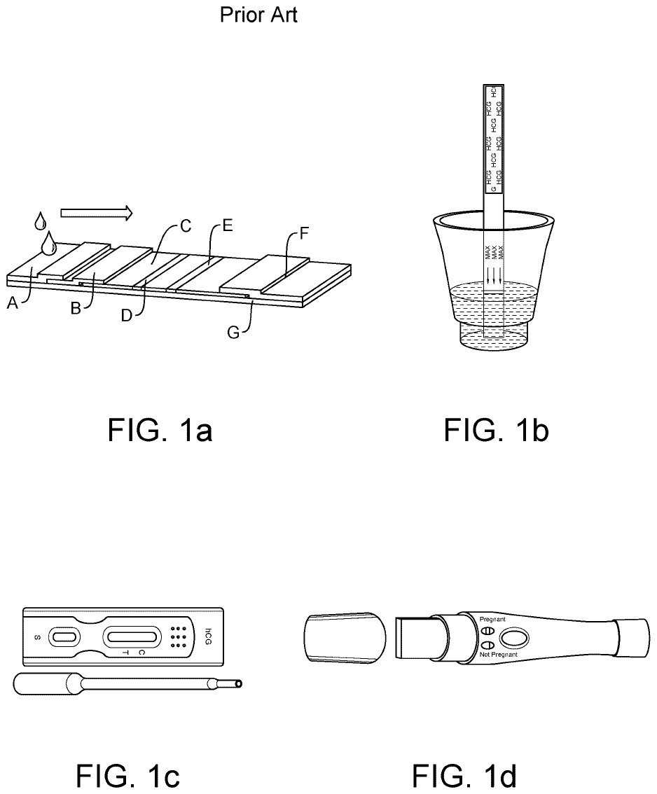

### DESCRIPTION OF THE INVENTION

Preferred embodiments of the invention will now be described with reference to the accompanying drawings and non-limiting examples.

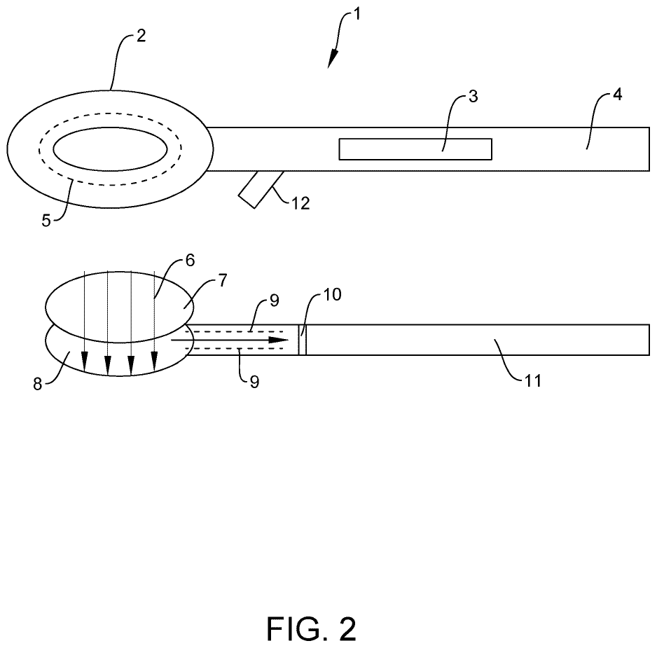

Referring to FIG. 2, there is provided an analyte detection device 1 comprising a sample collection reservoir 2, a test strip 3 and a handle 4. A preferred embodiment may comprise a wetness indicator 5 that may be placed, for example, around the perimeter of the sample collection reservoir 2 to indicate to the user when sufficient sample has been collected for the test to operate effectively. The downward arrows 6 shown over the sample pad 7 and conjugate pad 8 indicate the direction of sample fluid flow as the sample is collected in the sample collection reservoir 2. In particularly preferred embodiments of the device 1, path guides or embossing 9 is included to guide the direction of fluid flow from the sample collection reservoir 2. A polyvinyl chloride (PVC) dam 10 may also be included in testing area 11 to plug pores in the testing strip to encourage bonding of the test antibodies to the target analyte.

Also shown in FIG. 2 is flip out stand 12. In particularly preferred embodiments, a flip out stand is used on the back of the analyte detection device 1 to ensure that liquid is directed to the test strip 3.

The familiar and intuitive shape of the analyte detection device 1 according to the present invention is for an application such as a pregnancy test and is intended to provide confidence to the user in holding it while a urine sample is collected into the sample collection reservoir 2. The wide design of the sample collection reservoir 2 allows for both a comfortable user experience for the capture of a fluid sample and provides confidence in the amount, volume and directionality of fluid flow as it reaches desired points. This is achieved, for example, using the colour change of wetness indicator 5 in some preferred embodiments. This novel design allows for the processing of an increased volume of sample liquid to ensure that the test is run with sufficient sample for an accurate result. Further, test results appear in the test strip 3 in the handle of the device 4 to make it clear to the user where to place sample in the sample collection reservoir 2. As shown in FIG. 2, vertical and horizontal flow of the fluid through layered padding and retention of a greater volume of sample allows for improved control of fluid flow, timing of the analyte reaction with the test antibody, and ultimately sensitivity.

A typical rapid pregnancy test will detect a hormone, human chorionic gonadotropin (hCG). This hormone is produced by the placenta from the time at which the embryo attached to the uterus.⁵ The hCG can be detected in urine from about 9 days after fertilisation⁶. Urine is applied to a sample pad of a rapid pregnancy test and hCG will be present in the urine if the woman is pregnant. The hCG binds to mobile antibodies which also have another component such as an enzyme attached to them⁷. A second immobilised enzyme in the test zone will also bind to the hCG and the enzyme on the first antibody will change the test line colour, and excess antibodies will bind to the immobilised antibodies in the control zone to show the test worked correctly⁸. ⁵ https://www.compoundchem.com/2018/11/09/pregnancy-tests/⁶ Ibid⁷ Ibid⁸ Ibid

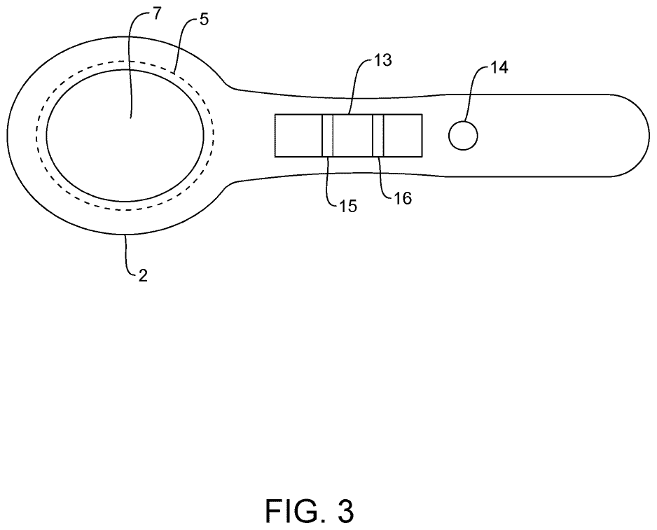

In the embodiment of FIG. 3, a test window 13 and additional control indicator 14 are shown in the handle 4 of the device 1. The additional control indictor 14 can be included for further visual control of the amount of sample liquid required, Test line 15 and control line 16 are shown in the test window 13. The spoon or ladle shaped design of the sample collection reservoir 2 means that the liquid samples such as urine are captured easily and sufficient sample is provided to the test line 15 and control line 16 to be able to provide a definite, accurate reading.

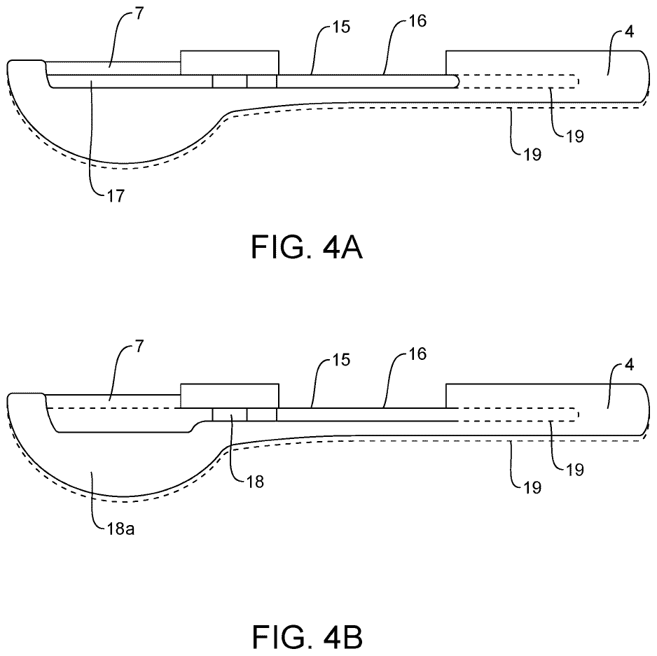

The preferred embodiments of FIG. 4A and FIG. 4B illustrate the placement of the conjugate release pad 8 closer to the fluid (adjacent the sample collection pad 7) without risking the loss of mobile labelling reagent and antibodies. In existing midstream tests with a wick, it is not possible to place the conjugate release pad at the site of sample collection because the antibody would ‘run off’ with the flow of fluid over the wick.

As shown in FIG. 4A, some embodiments of the analyte detection device 1 have the mobile labelling reagent contained within the sample collection reservoir 2, in a conjugate release layer 17 beneath the sample collection pad 7. The mobile labelling reagent is rehydrated before binding to the target analyte in the sample and then flowing together into the handle 4 of the device 1 to the test strip 3 consisting of test line 15 and control line 16.

As shown in FIG. 4B, some other embodiments of the analyte detection device 1 have the mobile labelling reagent contained within the handle 4 of the device 1. That is, conjugate release pad 18 is shown located in the handle 4 of the device 1. In some embodiments of the device, the area 18a visible in the spoon shaped reservoir in FIG. 4B is a material which can be manipulated to control the amount of liquid which is absorbed. That is, to further inhibit run-off by catching liquid, or impregnated with a hydrophobic substance to prevent absorption of liquid, and direct liquid into the handle of the device.

FIGS. 4A and 4B also show rigid support layer 19 which can be made from natural substances such as corn-starch, gelatine, or sugar. In certain embodiments, the rigid support layer 19 replaces the plastic backing of existing tests with a water-resistant backing made from a gelatine material, plant-based eco plastic, or a non-woven fibre that has been sealed with a substance that is rapidly dissolvable once it has come in contact with water.

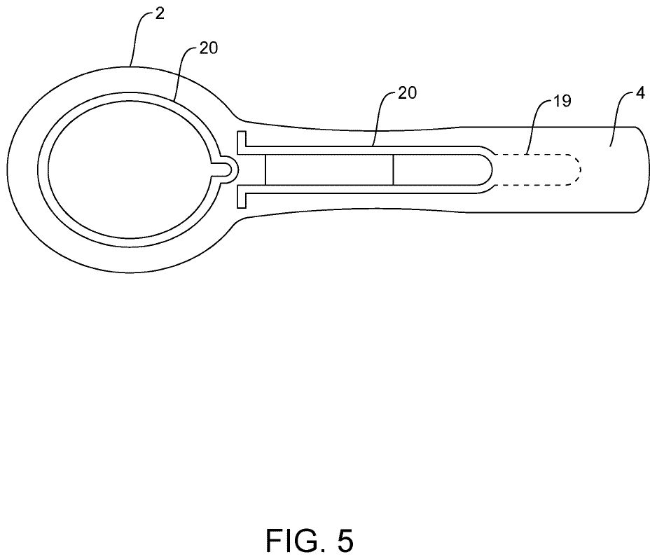

The preferred embodiment of FIG. 5 shows channels 20 which have been coated in wax or embossed to direct fluid flow. In the embodiment of FIG. 5, the direction and speed of the fluid can be controlled through the integration of ‘fabricated’ channels 20 with borders composed of a non-toxic material such as a wax or plant-based glue that help to guide the direction of liquid into the test strip 3 of the device 1.

Additionally, cut outs in the material may help to optimise the flow by slowing down, speeding up, or redirecting the volume of fluid to other areas of the test

Where fluid is not desired, the material of the device 1 may be further manipulated with a sugar, gelatine, or wax coating to control the direction of sample flow. For example, concave edges of the spoon or ladle shaped sample collection reservoir 2 will be coated to support flow of fluid into the centre of the device sampling zone.

As shown in FIG. 4A and FIG. 4B, coatings of sugar, gelatine or wax may also be used to support the rigidity of the device before and during use, for example under or on the handle 4 of the device 1 and the waterproof backing of the device.

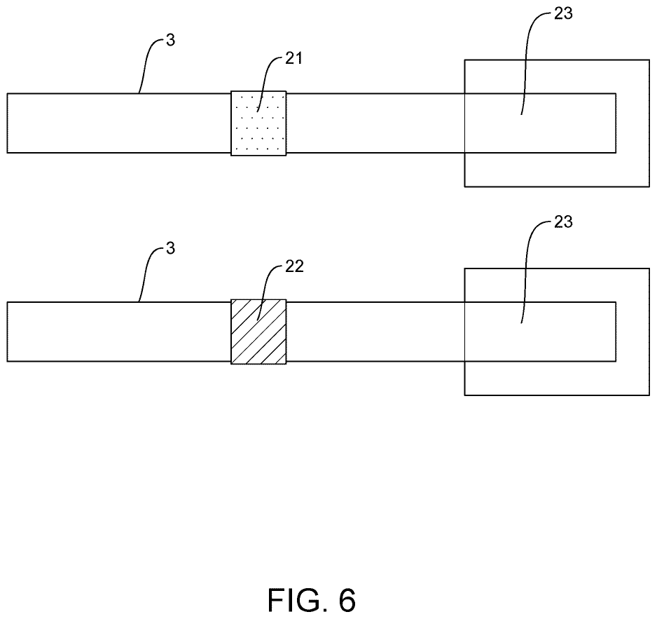

In the preferred embodiment of FIG. 6, PVC solution is dotted 21 on test strip 3 to support plugging of pores of the test strip to encourage reaction of the test antibody with the analyte of interest. In an alternative embodiment, PVC solution is coated 22 on test

strip to support plugging of pores. Region 23 on test strip 3 comprises the test line and control line. In further embodiments (not shown), no solution is used to plug the pores of the test strip. In still further embodiments, an alternative solution to PVC may be used to plug the pores of the test strip.

To further optimise sensitivity, some embodiments will incorporate a PVC (polyvinyl chloride) or other water-soluble substances which are rich in hydroxyl groups. As an illustration, the pore size of nitrocellulose, used in existing tests, is about 1×10⁻² or 0.01 or 10 microns. The pore size of cellulose is noticeably larger than nitrocellulose which impacts volume of flow and retention of substrates.

As shown in FIG. 6, in some preferred embodiments of the device of the present invention, a PVC dam is incorporated in order to plug the cellulose membrane pores and slow down the lateral flow of the analyte. This also has the benefit of leading to an increased reaction time between the analyte and the labelling reagent, and thus increased sensitivity because it improves the bio-recognition time. An important consideration in the development of the device of the present invention was slowing down the flow of the sample prior to the test zone and controlling for the time of reaction by incorporation of a substance such as PVC which upon contact with solution dissolves and plugs in the pores of the testing area to ensure slow dispersion.

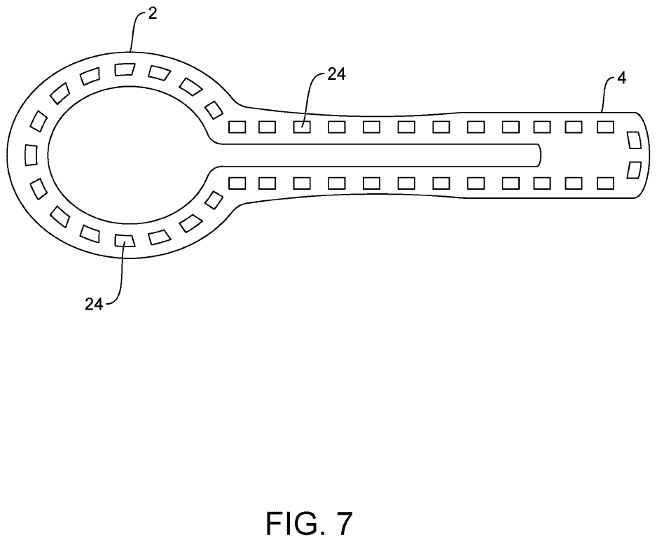

In the preferred embodiment of FIG. 7, wells 24 are shown around the perimeter of the device to be filled with dissolvable substance to facilitate dispersion/flushability of device;

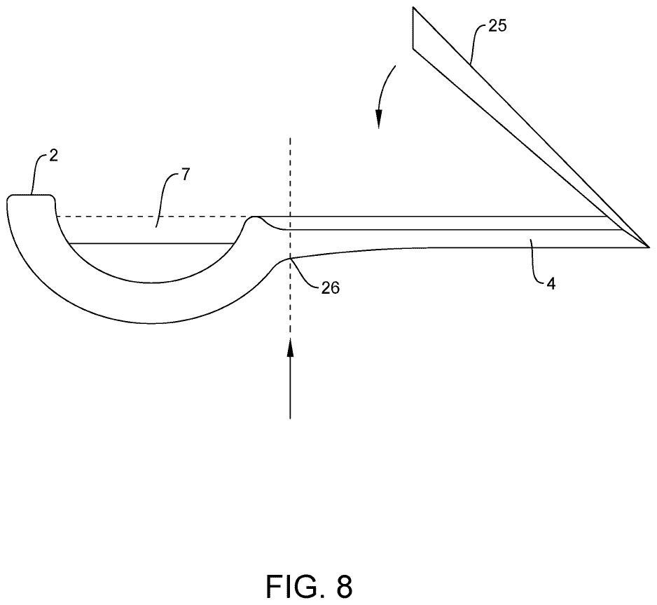

As shown in FIG. 8, discretion is incorporated into the design of the device 1. In some preferred embodiments of the device 1, a pouch is included to place the used device within and support the biodegradability/dissolution of the test. In some embodiments, it is possible to dissolve the device under the running water. In FIG. 8, a preferred embodiment of the device is shown wherein it is possible to fold the distal end of the handle 25 to support discretion. In some embodiments of the device, the sample reservoir 2 can be detached from the handle 4 at separation point 26. The test strip 3 with results can also be pulled off and flushed away.

In preferred embodiments of the present invention, the nitrocellulose membrane of existing tests is replaced with a cellulose membrane, and glass fibre conjugate and adsorption pads are replaced with a biodegradable blended cellulose membrane.

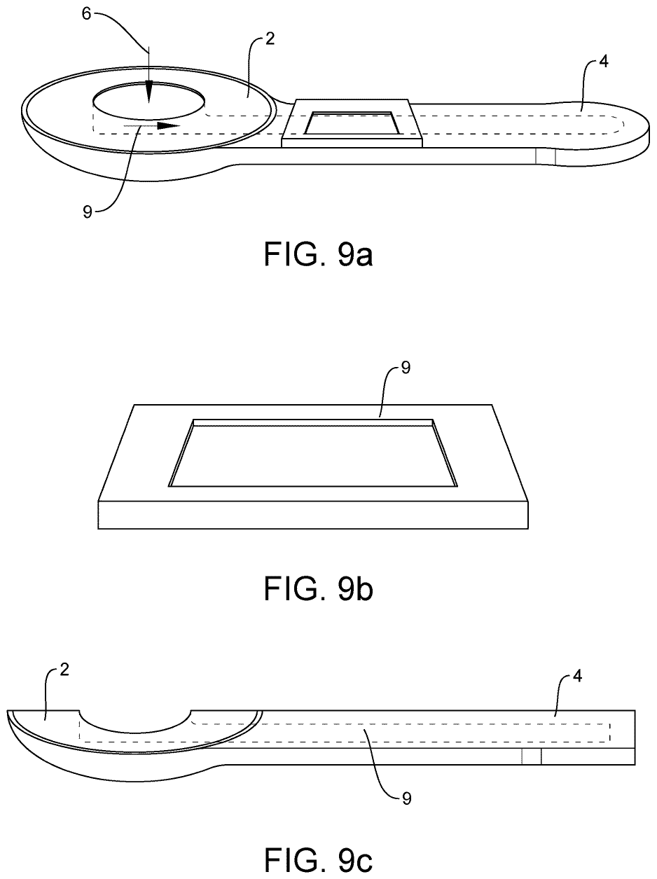

FIG. 9 shows greater detail of the vertical and horizontal fluid flow channels of FIG. 2. Downward arrows 6 indicate the direction of sample fluid flow as the sample is collected in the sample collection reservoir 2 and path guides, hydrophobic coating or embossing 9 are included to guide the direction of fluid flow from the sample collection reservoir 2. In FIG. 9B, a raised edge and a coating are included to support fluid flow across the testing window.

The device of the present invention is designed such that the device is readily biodegradable. However, the material of the device needs to be sufficiently durable and resist degradation long enough to hold a biochemical reaction during the time the urine is passing through into the sample collection pad 7 and conjugate release pad 8.

A key feature of the device of the present invention is enhanced antibody immobilization on cellulose through the development of a custom antibody, such as a monoclonal, single-chain variable fragment, full length or camelid nanobody fused to a molecule with an affinity to cellulose. Cellulose binding molecules such as carbohydrate-binding modules (CBMs) are fused to antibody fragments to increase protein binding capacity. In testing, a 2.7-fold protein binding capacity of CBM-fused antibody fragments is shown compared to the sole antibody fragment. It has been shown that improved spatial retention of antibodies to the test area leading to enhanced sensitivity and improved overall LFA-performance compared to the naked detection antibody.

The use of cellulose paper-based lateral flow immunoassays using a carbohydrate-binding module-fused to detection antibodies has been investigated in literature. For example, Elter et al. demonstrated the validation of CBM-assisted antibodies by implementation into two model lateral flow test devices (pregnancy detection and the detection of SARS-CoV-2 specific antibodies). The CBM-assisted pregnancy LFA demonstrated sensitive detection of human gonadotropin (hCG) in synthetic urine⁹. However, this research is relatively new and theoretically based using only synthetic urine. Hussack et al¹⁰. has demonstrated the feasibility of CBM-assisted antibodies linked via a nanobody platform, demonstrating only one hCG-specific nanobody targeting the alpha subunit of hCG, a subunit the hormone shares with a number of other hormones including LH and FSH. However, no hCG-specific nanobody for the beta subunit of hCG exists. There is no widely available commercial lateral flow immunoassay test that is available at the time of writing that utilises such technology. The “at home” pregnancy test has changed very little since the late-20th century even as technological innovations have emerged within the space. Innovations in women's health related products are often deprioritised and the status quo remains even when the experience is less than optimal from the perspective of ease of use, convenience, readability, accuracy, and sustainability. ⁹ Elter, A., Bock, T., Spiehl, D. et al. Carbohydrate binding module-fused antibodies improve the performance of cellulose-based lateral flow immunoassays. Sci Rep 11, 7880 (2021). https://doi.org/10.1038/s41598-021-87072-7¹⁰ Hussack G, Luo Y, Veldhuis L, Hall JC, Tanha J, Mackenzie R. Multivalent anchoring and oriented display of single-domain antibodies on cellulose. Sensors (Basel). 2009;9(7):5351-67. doi: 10.3390/s90705351.

Another key feature of the device of the present invention is improved sensitivity of assay through the utilisation of an unbleached wood pulp and viscose blend. Cellulose fibres are rich in hydroxyl groups that can have strong interactions with the sample, slowing down the sample flow. This controlled delayed sample flow gives more interaction time within the biomolecules and contributes to the enhanced sensitivity. Unbleached wood pulps maintained their dispersibility better than bleached wood pulp/viscose blends.

Preferred embodiments of the device of the present invention utilise eco-friendly materials that are biodegradable, compostable, and/or flushable. These materials include unbleached wood pulp, viscose, potato, sugar cane, wheat straw fibres, banana fibres, corn-starch or other biodegradable materials.

Another feature of the device of the present invention is the development of a custom antibody that is sensitive to hyperglycosylated hCG, the principal form of hCG present in early pregnancy urine (TBD). Cellulose binding molecules such as carbohydrate-binding modules (CBMs), or other such molecules with an affinity to cellulose, are a key feature of the invention for optimizing the binding to cellulose to develop a custom antibody for use in the present invention. Such a custom antibody is not available in commercial markets at the time of writing. The present invention is advantageously utilising cellulose binding molecules to improve upon the detection of women's health related hormones in a device that is widely available for use.

The device of the present invention may be used with a wide variety of molecules such as, for example, pregnanediol glucuronide (PDG), the urine metabolite of progesterone. In regards to hyperglycosylated hCG, only one antibody (B152) is currently available and this has not been optimised since initial production. Hyperglycosylated hCG has the same polypeptide structure as hCG, with much larger N-and O-linked oligosaccharides. Hyperglycosylated hCG is made from extravillous cytotrophoblast cells, and is an independent molecule from hCG with completely separate biological functions. The use of hyperglycosylated hCG may contribute to the sophistication of lateral flow immunoassay test design by, for example, being able to differentiate pregnancies that will miscarry, or to screen for Down Syndrome.

Another feature of the present invention is the use of camelid-derived nanobodies as an alternative to conventional antibodies, particularly hCG-specific nanobodies. The use of such recombinant nanobodies in the detection of women's health related hormones is not known in the commercial marketplace at the time of writing as there has been no motivation to innovate in this area of technology.

In certain embodiments of the present invention, the sample pad contained within the sample collection reservoir can be pre-treated with a range of other agents and enhancers to support optimal flow and signal readout such as blocking agents, detector reagent release agents, and viscosity enhancers.

For example, because differences in the pH of human urine can affect the specificity and sensitivity of capture and detector reagents, and result in non-specific binding of mobile labelling reagents, the material prior to the testing region can be treated with buffer salts, for example, to modify the pH and ionic strength.

Another feature of the device of the present invention is improved dispersibility through use of a custom designed fibre. The material of the device of the present invention is intended to have a flat cross section as it has been shown that when utilising the same pulp, viscose fibre A, which has a flat cross section, provides better dispersibility than viscose fibre B with a circular cross section. This has also been observed that the improved dispersibility had been attributed to the better access of shear forces to the flat viscose fibres during disintegration. While not being bound by theory, it is understood that the long viscose fibres form the load-carrying structure providing wet strength and the pulp fibres attached to the body are responsible for liquid absorption and dispersive properties.

The novel design of the present invention makes use of vertical and horizontal fluid flow through the device with layered textiles/padding. For example, in some embodiments of the device of the invention, when fluid enters the device, it is directed through an initial sample pad, to a conjugate pad where reaction and binding to analyte can take place prior to flowing through the handle. Novel control of the solution flow rate is also included to increase the interaction time for binding of the target analyte and test antibody by the inclusion of a ‘dam’ which enhances sensitivity and readout, for example through the use of polyvinyl chloride (PVC) or polydimethylsiloxane.

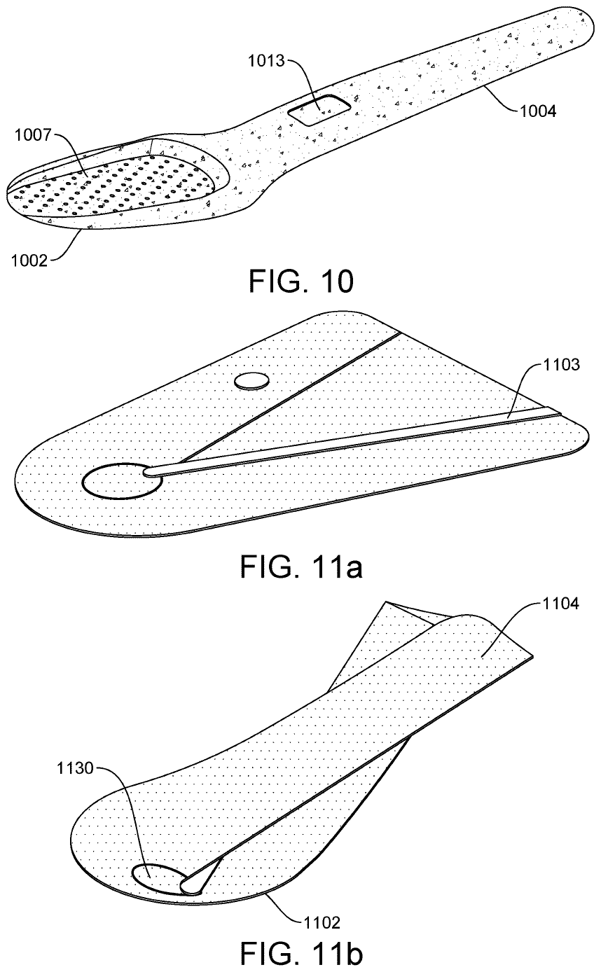

An alternative preferred embodiment is shown in FIG. 10. This embodiment has a spoon-like shape and is fully formed of a recyclable and biodegradable material, such as Notpla paper being a seaweed-based product. The collection reservoir 1002 and handle 1004 are coated with a suitable water-resistant material. A non-coated region 1007 located in the collection reservoir 1002 absorbs fluid to be tested and is in fluid communication with a test strip 3 which is embedded within the handle 1004. Ideally, the non-coated region changes colour while absorbing fluid in order to provide a visual indictor as to when sufficient fluid has been collected. A test window 1013 is provided on the handle 1004. This embodiment is designed to be fully compostable after use.

Another alternative preferred embodiment is shown in FIGS. 11a and 11b. As shown in FIG. 11a, the product is formed in a flat configuration, which is ideal for storage and packaging. This form also makes the product suitable for mass manufacture in the form of a continuous web of products. A test strip 1103 is provided. The product is formed of cellulose materials, as described earlier, which allows the product to be flushable for disposal after use. The flat configuration is designed to be folded into an in-use configuration, as shown in FIG. 11b. When folded, a collection reservoir 1102 and a handle portion 1104 are formed. An end of the test strip 1103 is in fluid communication with the collection reservoir 1102. Ideally, as shown, the test strip 1103 is hidden within the handle portion 1104. Consequently, to view the outcome of the test, the device is required to be unfolded so that the test strip is visible. This adds an element of surprise as to what the test outcome may be. Ideally, a bottom portion 1130 of the collection reservoir 1102 is designed to start dissolving upon contact with fluid to be tested. This acts as a visual indicator to the user to indicate that sufficient fluid has been collected when the bottom portion has completely dissolved.

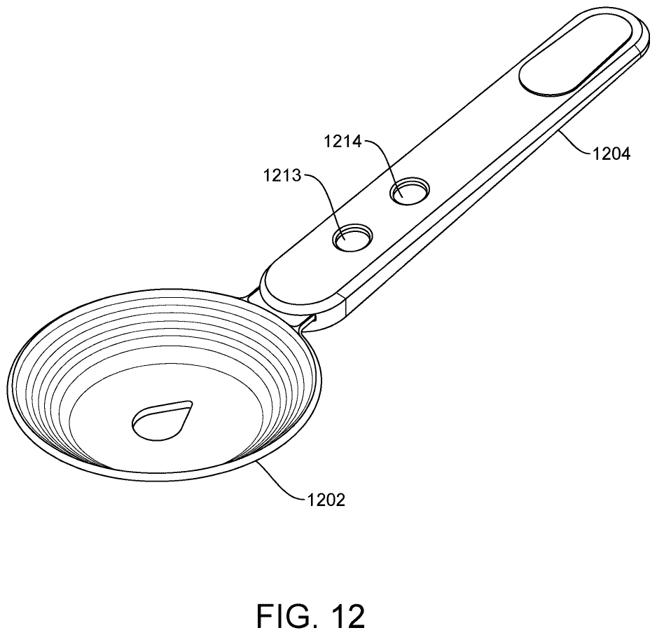

Another preferred embodiment is shown in FIG. 12. This embodiment has a spoon-like shape. The handle 1204, having an embedded test strip, is formed of a compostable material, such as a paper foam. A slot at an end of the handle 1204, receives the collection reservoir 1202 and bring the test strip into fluid communication with the collection reservoir 1202. The collection reservoir 1202 is formed of a dissolvable material, such as PVOH (Polyvinyl alcohol) and is designed to start dissolving upon contact with fluid to be tested. This acts as a visual indicator to the user to indicate that sufficient fluid has been collected when the collection reservoir 1202 has completely dissolved and only the handle 1204 remains. The handle has a test window 1213 and a control indicator 1214.

### FUTURE VARIATIONS

Further variations to preferred embodiments of the device of the present invention include:
Layering of cellulose padding;
Inclusion of vertical wells; Cutouts; Dots.
Proposed future variations on the device of the present invention are as follows:
Basic design: Biodegradable and Recyclable
Premium product: Flushable
Custom antibody innovations to further support stability and sensitivity
Hybrid device design
Design to allow for various strips to be added onto the device
Foldable design to support discretion
Easy tear off and flushable test region
Test can be placed in packaging which dissolves it
Making the spoon shaped sample reservoir detachable
Making the spoon shaped sample reservoir reusable such that the testing strip can be slotted into the device. This means that the device itself (made from some type of environmentally friendly material) would serve as the “backing”/hard substrate

required to allow the test to lay flat while reaction is taking place.

The key innovations of the device of the present invention are considered to be Intuitive design; Fluid volume control; Optimised microfluidics; One step handling; Rapid results; Discretion; Novel optimisation of binding to the cellulose paper through the use of a fusion protein; Indication of wetness.

The device of the present invention results in an improved urine test which will be appealing to end users, governments, and trusted by healthcare providers.

### POTENTIAL APPLICATIONS

People can test themselves for pregnancy, fertility, sexually transmitted diseases, vitamin D deficiency, food allergens, high levels of cholesterol, biomarkers related to diabetes or renal failure, or for cardiac markers that might indicate heart disease. Plus, menopause, thyroid health and enzyme indicators. As an added bonus these tests can be taken at home which saves public money and puts people in control of their own health and wellbeing.

There is also the use of lateral flow tests in the environment, such as soil analysis, testing pathogens in water, plant health, lead testing and Legionella tests. These uses mean a quick turnaround in terms of results, rather than samples being sent to labs.

Relevant sample fluids may include:
Biological fluids such as urine, blood, serum, saliva and many more. Analytes of interest may include:
Hormones (e.g. hCG, LH, PDG, insulin) and other biomarkers such as proteins, peptides, vitamins, toxins, organic compounds, amino acid, drug, a metabolite, glucose, ketones, pH levels, bilirubin, nitrites/leukocyte esterase, alcohol, amphetamines, benzodiazepines, cannabis, cocaine, opioids, vitamin D, pathogens, soil analysis, pesticide detection, lead, plant health tests, legionella, menopause, thyroid, and enzyme indicators.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A device for detecting an analyte in a sample, the device comprising:
a test strip fluid including a sample collection pad for contacting the sample; and
a conjugate release pad in fluid communication with the sample collection pad and a test strip, wherein:
the conjugate release pad includes at least one labelling reagent,
the at least one labelling reagent comprises a component configured to bind to the analyte at a first location;
the test strip comprises a test line including a test antibody, and
the test antibody is configured to bind the analyte at a second location.

2. The device of claim 1, wherein the test strip comprises a cellulose membrane.

3-23. (canceled)

24. The device of claim 2, wherein the test strip includes no nitrocellulose materials.

25. The device of claim 1, wherein the sample collection pad includes a sample collection reservoir configured to collect the sample and guide flow of the sample to the conjugate release pad.

26. The device of claim 25, wherein the sample collection reservoir is oval.

27. The device of claim 25, wherein a length of the sample collection reservoir is greater than a width of the sample collection reservoir.

28. The device of claim 25, further comprising a handle configured to receive the test strip.

29. The device of claim 28, wherein each of the handle and the sample collection reservoir comprise only biodegradable and/or compostable materials.

30. The device of claim 29, wherein the biodegradable and/or compostable materials include at least one of unbleached wood pulp, vicose, potato, sugar cane, wheat straw fibres, banana fibres, corn-starch or cellulose.

31. The device of claim 28, further comprising:
a backing coupled to a first surface of the test strip opposite the sample collection pad; and
a supporting material configured to provide rigid support to the handle and/or the backing, wherein the supporting material comprises one or more of sugar, gelatine or wax.

32. The device of claim 25, further comprising:
a coating coupled to the sample collection reservoir, wherein the coating comprises one or more of sugar, gelatine or wax.

33. The device of claim 1, wherein the conjugate release pad comprises:
at least one vertical fluid flow path;
at least one horizontal fluid flow path; and
a plurality of fabricated channels with borders, wherein the plurality of fabricated channels with borders are configured to control a speed and a direction of the sample flowing through the at least one vertical fluid flow path and the at least one horizontal fluid flow path.

34. The device of claim 33, wherein the plurality of fabricated channels with borders are formed of a non-toxic material.

35. The device of claim 34, wherein the non-toxic material comprises at least one of a wax or a plant-based glue.

36. The device of claim 1, wherein the conjugate release pad comprises a blended cellulose membrane.

37. The device of claim 1, wherein the sample collection pad comprises cellulose fibres.

38. The device of claim 1, further comprising:
a backing coupled to a first surface of the test strip opposite the sample collection pad, wherein the backing comprises at least one of: a gelatine material, a plant-based eco plastic or a non-woven fibre.

39. The device of claim 1, wherein the device is configured to dissolve in a fluid.

40. The device of claim 1, wherein the device is biodegradable.
